(19) United States
(12) Patent Application Publication
(10) Pub. No.: US 2006/0210542 A1
(43) Pub. Date: Sep. 21, 2006

(54) Use of TPO mimetic compounds and pharmaceutical compositions in the treatment of anemia

(76) Inventors: Edward J. Yurkow, Hillsborough, NJ (US); Brian R. MacDonald, Newtown Square, PA (US); Jeffery K. Weis, Whitehouse Station, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/181,049

(22) Filed: Jul. 14, 2005

Related U.S. Application Data
(60) Provisional application No. 60/601,921, filed on Aug. 16, 2004.

(51) Int. Cl. A61K 35/14; A61K 38/18

(52) U.S. Cl. 424/93.7; 514/12

(57) ABSTRACT

A method of treating anemia is disclosed. The method includes administering a TPO mimetic compound to a subject. Pharmaceutical compositions including a TPO mimetic compound and a pharmaceutically acceptable carrier as well as diagnostic methods employing a labeled TPO mimetic compound are also disclosed.

### CROSS-REFERENCE TO RELATED APPLICATIONS

This application claims priority to Application No. 60/601,921 filed on Aug. 16, 2004.

### BACKGROUND OF THE INVENTION

The present invention provides peptides and compounds that bind to and activate the thrombopoietin receptor (c-mpl or TPO-R) or otherwise act as a thrombopoietin (“TPO”) agonist. The invention has application in the fields of biochemistry and medicinal chemistry and particularly provides TPO agonists for use in the treatment of human disease. The peptides and compounds of the invention may be used to prevent the development of anemia and to maintain normal production of red blood cells.

The gene encoding TPO has been cloned and characterized. See Kuter et al. Proc. Natl. Acad. Sci. USA 91:11104-11108 (1994); Barley et al. Cell 77:1117-1124 (1994); Kaushansky et al. Nature 369:568-571 (1994); Wendling et al. Nature 369:571-574 (1994); and Sauvage et al. Nature 369:533-538 (1994). TPO is a glycoprotein with at least two forms, with apparent molecular masses of 25 kDa and 31 kDa, with a common N-terminal amino acid sequence. See, Bartley et al. Cell 77:1117-1124 (1994). TPO appears to have two distinct regions separated by a potential Arg-Arg cleavage site. The amino-terminal region is highly conserved in man and mouse, and has some homology with erythropoietin and interferon-a and interferon-b. The carboxy-terminal region shows wide species divergence.

The DNA sequences and encoded peptide sequences for human TPO-R (also known as c-mpl) have been described. See Vigon et al. Proc. Natl. Acad. Sci. USA 89:5640-5644 (1992). TPO-R is a member of the haematopoietin growth factor receptor family, a family characterized by a common structural design of the extracellular domain, including four conserved C residues in the N-terminal portion and a WSXWS motif (SEQ ID NO:1) close to the transmembrane region. See Bazan Proc. Natl. Acad. Sci. USA 87:6934-6938 (1990). Evidence that this receptor plays a functional role in hematopoiesis includes observations that its expression is restricted to spleen, bone marrow, or fetal liver in mice (see Souyri et al. Cell 63:1137-1147 (1990)) and to megakaryocytes, platelets, and CD34⁺ cells in humans (see Methia et al. Blood 82:1395-1401 (1993)). Some workers postulate that the receptor functions as a homodimer, similar to the situation with the receptors for G-CSF and erythropoietin.

The availability of cloned genes for TPO-R facilitates the search for agonists of this important receptor. The availability of the recombinant receptor protein allows the study of receptor-ligand interaction in a variety of random and semi-random peptide diversity generation systems. These systems are disclosed in U.S. Pat. Nos. 6,251,864, 6,083,913, 6,121,238, 5,932,546, 5,869,451, 6,506,362, and 6,465,430, and in Cwirla et al., Proc. Natl. Acad. Sci. USA 87:6378-6382 (1990), each of the foregoing is incorporated herein by reference.

The morphologically recognizable and functionally capable cells circulating in blood include erythrocytes, neutrophilic, eosinophilic, and basophilic granulocytes, B-, T-, non B-, non T-lymphocytes, and platelets. These mature hematopoietic cells derive from and are replaced, on demand, by morphologically recognizable dividing precursor cells for the respective lineages such as erythroblasts for the erythrocytes series, myeloblasts, promyelocytes and myelocytes for the granulocyte series, and megakaryocytes for the platelets. The precursor cells derive from more primitive cells that can simplistically be divided into two major subgroups: stem cells and progenitor cells (for review, see Broxmeyer, H. E., 1983, “Colony Assays of Hematopoietic Progenitor Cells and Correlations to Clinical Situations,” CRC Critical Review in Oncology/Hematology 1:227-257).

The definitions of stem and progenitor cells are operational and depend on functional, rather than on morphological, criteria. Stem cells have extensive self-renewal or self-maintenance capacity (Lajtha, Differentiation, 14:23 (1979)), a necessity since absence or depletion of these cells could result in the complete depletion of one or more cell lineages, events that would lead within a short time to disease and death. Some of the stem cells differentiate upon need, but some stem cells produce other stem cells to maintain the pool of these cells. Thus, in addition to maintaining their own kind, pluripotential stem cells are capable of differentiation into several sub-lines of progenitor cells with more limited self-renewal capacity or no self-renewal capacity. These progenitor cells ultimately give rise to the morphologically recognizable precursor cells. The progenitor cells are capable of proliferating and differentiating along one, or more than one, of the myeloid differentiation pathways (Lajtha, Blood Cells, 5:447 (1979)).

A variety of infectious agents, genetic abnormalities and environmental factors can cause a deficiency in one or more hematopoietic cell types. Additionally, chemotherapy and radiation therapy used in the treatment of cancer and certain immunological disorders can cause pancytopenias or combinations of anemia, neutropenia and thrombocytopenia. Thus, the increase or replacement of hematopoietic cells is often crucial to the success of such treatments. (For a general discussion of hematological disorders and their causes, see, e.g., “Hematology” in Scientific American Medicine, E. Rubenstein and D. Federman, eds., Volume 2, Chapter 5, Scientific American, New York (1996)).

The current therapy available for many hematological disorders as well as the destruction of the endogenous hematopoietic cells caused by chemotherapy or radiotherapy is bone marrow transplantation. However, use of bone marrow transplantation is severly restricted since it is extremely rare to have perfectly matched (genetically identical) donors, except in cases where an identical twin is available or where bone marrow cells of a patient in remission are stored in a viable frozen state. Except in such autologous cases, there is an inevitable genetic mismatch of some degree, which entails serious and sometimes lethal complications. These complications are two-fold. First, the patient is usually immunologically incapacitated by drugs beforehand, in order to avoid immune rejection of the foreign bone marrow cells (host versus graft reaction). Second, when and if the donated bone marrow cells become established, they can attack the patient (graft versus host disease), who is recognized as foreign. Even with closely matched family donors, these complications of partial mismatching are the cause of substantial mortality and morbidity directly due to bone marrow transplantation from a genetically different individual.

Peripheral blood has also been investigated as a source of stem cells for hematopoietic reconstitution (Nothdurtt, W., et al., 1977, Scand. J. Haematol. 19:470-481 ; Sarpel, S. C., et al., 1979, Exp. Hematol. 7:113-120; Ragharachar, A., et al., 1983, J. Cell. Biochem. Suppl. 7A:78; Juttner, C. A., et al., 1985, Brit. J. Haematol. 61:739-745; Abrams, R. A., et al., 1983, J. Cell. Biochem. Suppl. 7A:53; Prummer, O., et al., 1985, Exp. Hematol. 13:891-898). In some studies, promising results have been obtained for patients with various leukemias (Reiffers, J., et al., 1986, Exp. Hematol. 14:312-315; Goldman, J. M., et al., 1980, Br. J. Haematol. 45:223-231; Tilly, H., et al., Jul. 19, 1986, The Lancet, pp. 154-155; see also To, L. B. and Juttner, C. A., 1987, Brit. J. Haematol. 66: 285-288, and references cited therein); and with lymphoma (Korbling, M., et al., 1986, Blood 67:529-532). Other studies using peripheral blood, however, have failed to effect reconstitution (Hershko, C., et al., 1979, The Lancet 1:945-947; Ochs, H. D., et al., 1981, Pediatr. Res. 15:601). Studies have also investigated the use of fetal liver cells transplantation (Cain, G. R., et al., 1986, Transplantation 41:32-25; Ochs, H. D., et al., 1981, Pediatr. Res. 15:601; Paige, C. J., et al., 1981, J. Exp. Med. 153:154-165; Touraine, J. L., 1980, Excerpta Med. 514:277; Touraine, J. L., 1983, Birth Defects 19:139; see also Good, R. A., et al., 1983, Cellular Immunol. 82:44-45 and references cited therein) or neonatal spleen cell transplantation (Yunis, E. J., et al., 1974, Proc. Natl. Acad. Sci. U.S.A. 72:4100) as stem cell sources for hematopoietic reconstitution. Cells of neonatal thymus have also been transplanted in immune reconstitution experiments (Vickery, A. C., et al., 1983, J. Parasitol. 69(3):478-485; Hirokawa, K., et al., 1982, Clin. Immunol. Immunopathol. 22:297-304).

Clearly, there is a tremendous need for methods of expanding blood cells in vitro or therapies which increase the production of hematopoietic cells in vivo.

Anemia is defined as a reduction in the hemoglobin concentration of the blood, usually associated with a reduction of total circulating red cell mass. Regardless of the cause, anemia decreases the oxygen-carrying capacity of the blood, and when severe enough, causes clinical symptoms and signs.

Clinically, anemia is characterized by pallor of the skin and mucus membranes, and by manifestations of hypoxia, most commonly weakness, fatigue, lethargy, or dizziness. Myocardial hypoxia may produce hyperdynamic circulation with an increase in heart rate and stroke volume. Ejection type flow murmurs may develop, and if the anemia is severe enough, cardiac failure may ensue.

Anemias are generally classified in one of two ways: either by etiological classification (based on the cause) or by morphologic classification (based on changes in shape and size). Etiological classification is more commonly employed.

Alloimmune hemolytic anemia occurs when the antibody of one individual reacts with red blood cells (RBC) of another. Alloimmune hemolytic anemia typically occurs following transfusion of ABO incompatible blood and rhesus disease of the newborn. It also can occur following allogenic transplantation. [Hoffbrand, A. V. in Essential Hematology, 3rd. ed., Blackwell Scientific Publications, 1993, p. 90].

The administration of certain drugs can cause transient drug induced anemia. This can occur by three mechanisms: 1) antibody directed against a drug-red cell membrane complex (e.g., penicillin or cephalothin); 2) deposition of complement via drug-protein (antigen)-antibody complex onto the red cell surface (e.g., quinidine or chloropropamide) or 3) an autoimmune-hemolytic anmeia in which the role of the drug is unknown (e.g., methyl dopa). In each case, the anemia disappears only after the drug is discontinued (however, with methyl dopa, the antibodies may persist for many months). [Hoffbrand, A. V. in Essential Hematology, 3rd. ed., Blackwell Scientific Publications, 1993, p. 90-1].

Aplastic anemia is defined as pancytopenia (anemia, leucopenia, and thrombocytopenia) resulting from aplasia of the bone marrow. It is classified into primary types: a congenital form (Fanconi anemia) and an acquired form with no obvious precipitating cause (idiopathic). Secondary causes may result from a variety of industrial, iatrogenic and infectious causes. The underlying cause appears to be a substantial reduction in the number of hemopoietic pluripotential stem cells and a defect in the remaining stem cells or an immune reaction against them making them unable to divide and differentiate sufficiently to populate the bone marrow. [Hoffbrand, A. V. in Essential Hematology, 3rd. ed., Blackwell Scientific Publications, 1993, p. 121]. Suppresser T-cells cells as well as immunoglobulins that inhibit erythropoietin or block differentiation of hemopoietic stem cells in vitro have been demonstrated in some cases. [Andreoli, T. in Essentials of Medicine, W. B. Saunders, 1986, p. 349].

Neelis et al., Blood, 90(1):58-63 (1997), discloses that human recombinant TPO stimulated red blood cell lineage recovery in rhesus monkeys exposed to 5 Gy total body irradiation (300-kV x-rays), with reticulocyte regeneration being initiated 10 days earlier than in placebo-treated animals. Neelis et al. also discloses improved hemoglobin and hematocrit values than in controls.

Basser et al., Blood, 89(9):3118-3128 (1997), discloses that administration of PEG-rHuMGDF plus filgastrim elevated peripheral blood progenitor cells of patients exposed to carboplatin 600 mg/m2 and cyclophosphamide 1,200 mg/m2.

Papayannopoulou et al., Exp. Hematol., 24(5):660-669 (1996), discloses the effects of EPO and TPO on the in vitro differentiation toward erythropoiesis and thrombopoiesis.

Kaushansky et al., J. Clih. Invest., 96(3): 1683-1687 (1995), discloses that TPO acted in synergy with EPO to expand erythroid progenitors. Kaushansky et al., Exp. Hematol., 24(2):265-269 (1996), discloses that TPO expanded BFU-E, CFU-GM and CFU-Mk progenitor cells in myelosuppressed animals.

Anemia is a serious problem, and has lent urgency to the search for a blood growth factor agonist able to prevent the development of anemia, promote the survival of RBC precursors and to maintain the normal production of red blood cells. The present invention provides such an agonist.

### SUMMARY OF THE INVENTION

The present invention is directed to defined low molecular weight peptides and peptide mimetics that have strong binding properties to the TPO-R, can activate the TPO-R and have the ability to stimulate, in vivo and in vitro, the production of red blood cells. The low molecular weight peptides and peptide mimetics can be in various forms, e.g., monomers, dimmers and oligomers. Accordingly, such peptides and peptide mimetics are useful for therapeutic purposes in treating anemia as well as for diagnostic purposes in studying anemia.

Peptides and peptide mimetics suitable for therapeutic and/or diagnostic purposes have an IC₅₀ of about 2 mM or less, wherein a lower IC₅₀ correlates to a stronger binding affinity to TPO-R. For pharmaceutical purposes, the peptides and peptidomimetics preferably have an IC₅₀ of no more than about 100 μm, more p referably, no more than about 500 nM, more preferably, no more than about 100 pm, and more preferably about 5 pm. In a preferred embodiment, the molecular weight of the peptide or peptide mimetic is from about 250 to about 8000 daltons.

Accordingly, preferred peptides and peptide mimetics comprise a compound having:
(1) a molecular weight of less than about 5000 daltons, and
(2) a binding affinity to TPO-R as expressed by an IC₅₀ of no more than about 100 μm.

When used for diagnostic purposes, the peptides and peptide mimetics preferably are labeled with a detectable label and, accordingly, the peptides and peptide mimetics without such a label serve as intermediates in the preparation of labeled peptides and peptide mimetics.

Peptides meeting the defined criteria for molecular weight and binding affinity for TPO-R comprise 9 or more amino acids wherein the amino acids are naturally occurring or synthetic (non-naturally occurring) amino acids. Peptide mimetics include peptides having one or more of the following modifications:
peptides wherein zero or more of the peptidyl [—C(O)NR—] linkages (bonds) have been replaced by a non-peptidyl linkage such as a —CH₂-carbamate linkage [—CH₂—OC(O)NR—]; a phosphonate linkage; a —CH₂-sulfonamide [—CH₂ —S(O)₂ NR-] linkage; a urea [—NHC(O)NH—] linkage; a —CH₂-secondary amine linkage; or an alkylated peptidyl linkage [—C(O)NR⁶—where R⁶ is lower alkyl];
peptides wherein the N-terminus is derivatized to a —NRR¹ group; to a —NRC(O)R group; to a —NRC(O)OR group; to a —NRS(O)₂ R group; to a —NHC(O)NHR group where R and R¹ are hydrogen or lower alkyl with the proviso that R and R¹ are not both hydrogen; to a succinimide group; to a benzyloxycarbonyl—NH—(CBZ—NH—) group; or to a benzyloxycarbonyl—NH—group having from 1 to 3 substituents on the phenyl ring selected from the grout consisting of lower alkyl, lower alkoxy, chloro, and bromo; or
peptides wherein the C terminus is derivatized to —C(O)R where R² is selected from the group consisting of lower alkoxy, and —NR³ R⁴ where R³ and R⁴ are independently selected from the group consisting of hydrogen and lower alkyl.

In some embodiments of the invention, preferred peptides for use include peptides having a core structure comprising a sequence of amino acids (SEQ ID NO:2):
X₁X₂X₃X₄X₅X₆X₇
where X₁ is C, L, M, P, Q, V; X₂ is F, K, L, N, Q, R, S, T or V; X₃ is C, F, I, L, M, R, S, V or W; X₄ is any of the 20 genetically coded L-amino acids; X₅ is A, D, E, G, K, M, Q, R, S, T, V or Y; X₆ is C, F, G, L, M, S, V, W or Y; and X₇ is C, G, I, K, L, M, N, R or V.

In a preferred embodiment the core peptide comprises a sequence of amino acids (SEQ ID NO:3):
X₈G X₁X₂X₃X₄X₅W X₇
where X₁ is L, M, P, Q. or V; X₂ is F, R, S, or T; X₃ is F, L, V, or W; X₄ is A, K, L, M, R, S, V, or T; X₅ is A, E, G, K, M, Q, R, S, or T; X₇ is C, I, K, L, M or V; and each X₈ residue is independently selected from.any of the 20 genetically coded L-amino acids, their stereoisomeric D-amino acids; and non-natural amino acids. Preferably, each X₈ residue is independently selected from any of the 20 genetically coded L-amino acids and their stereoisomeric D-amino acids. In a preferred embodiment (SEQ ID NO:4), X₁ is P; X₂ is T; X₃ is L; X₄ is R; X₅ is E or Q; and X₇ is I or L.

More preferably, the core peptide comprises a sequence of amino acids (SEQ ID NO:5):
X₉X₈G X₁X₂X₃X₄X₅W X₇
where X₉ is A, C, E, G, I, L, M, P, R, Q, S, T, or V; and X₈ is A, C, D, E, K, L, Q, R, S, T, or V. More preferably, X₉ is A or I; and X₈ is D, E, or K.

Particularly preferred peptides include (SEQ ID NOS:6-13, respectively): G G C A D G P T L R E W I S F C G G; G N A D G P T L R Q W L E G R R P K N; G G C A D G P T L R E W I S F C G G K; T I K G P T L R Q W L K S R E H T S; S I E G P T L R E W L T S R T P H S; L A I E G P T L R Q W L H G N G R D T; C A D G P T L R E W I S F C; and I E G P T L R Q W L A A R A.

A preferred TPO mimetic peptide is a PEGylated 29-mer peptide having 2 identical 14-mers linked by a lysinamide residue. A particularly preferred TPO mimetic peptide is:
I E G P T L R Q (2-Nal)L A A R A(SEQ ID NO:14).

In another embodiment, the TPO mimetic peptide is dimerized or oligomerized to increase the affinity and/or activity of the compound. An example of such a compound includes: 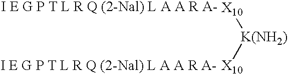
where X₁₀ is a sarcosine or β-alanine residue or a pegylated form of this compound (SEQ ID NO:15). The pegylated form may include a 20 k MPEG residue covalently linked to each N-terminal isoleucine. This compound is referred to herein as Compound I.

One or more TPO mimetic peptides, and in particular PEGylated TPO mimetic peptides (collectively referred to herein as “TPO mimetic compounds” or “TPO mimetic compounds of the invention”), are useful for the prevention and treatment of diseases mediated by TPO, and particularly for treating anemia. Thus, the present invention also provides a method for treating anemia, wherein a patient having anemia receives, or is administered, a therapeutically effective dose or amount of a compound of the present invention.

The invention also provides for pharmaceutical compositions comprising one or more of the compounds described herein and a physiologically acceptable carrier. These pharmaceutical compositions can be in a variety of forms including oral dosage forms, as well as inhalable powders and solutions and injectable and infusible solutions.

### BRIEF DESCRIPTION OF THE FIGURES

FIG. 1 shows the effect of treatment of Compound I on hemoglobin levels as set forth in Example 1.

FIG. 2 shows the effect of treatment of Compound I on red blood cell count as set forth in Example 1.

FIG. 3 shows the effect of treatment of Compound I on hematocrit as set forth in Example 1.

FIG. 4 shows the effect of treatment of Compound I on body weight as set forth in Example 1.

FIG. 5 shows the effect of treatment of Compound I on hemoglobin levels as set forth in Example 2.

FIG. 6 shows the effect of treatment of Compound I on red blood cell count as set forth in Example 2.

FIG. 7 shows the effect of treatment of Compound I on hematocrit as set forth in Example 2.

FIG. 8 shows the effect of treatment of Compound I on body weight as set forth in Example 2.

FIG. 9 shows the effect of treatment of Compound I on hemoglobin levels as set forth in Example 3.

FIG. 10 shows the effect of treatment of Compound I on red blood cell count as set forth in Example 3.

FIG. 11 shows the effect of treatment of Compound I on hematocrit as set forth in Example 3.

FIG. 12 shows the effect of treatment of Compound I on body weight as set forth in Example 3.

FIG. 13 shows the effect of treatment of Compound I on hematocrit as set forth in Example 4.

FIG. 14 shows the effect of treatment of Compound I on body weight as set forth in Example 4.

FIG. 15 shows the effect of of treatment of Compound I on body weight as set forth in Example 5.

### DESCRIPTION OF SPECIFIC EMBODIMENTS

The following definitions are set forth to illustrate and define the meaning and scope of the various terms used to describe the invention herein.

“Agonist” refers to a biologically active ligand which binds to its complementary biologically active receptor and activates the latter either to cause a biological response in the receptor or to enhance preexisting biological activity of the receptor.

“Pharmaceutically acceptable salts” refer to the non-toxic alkali metal, alkaline earth metal, and ammonium salts commonly used in the pharmaceutical industry including the sodium, potassium, lithium, calcium, magnesium, barium, ammonium, and protamine zinc salts, which are prepared by methods well known in the art. The term also includes non-toxic acid addition salts, which are generally prepared by reacting the compounds of this invention with a suitable organic or inorganic acid. Representative salts include the hydrochloride, hydrobromide, sulfate, bisulfate, acetate, oxalate, valerate, oleate, laurate, borate, benzoate, lactate, phosphate, tosylate, citrate, maleate, fumarate, succinate, tartrate, napsylate, and the like.

“Pharmaceutically acceptable acid addition salt” refers to those salts which retain the biological effectiveness and properties of the free bases and which are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, menthanesulfonic acid, ethanesulfonic acid, p-toluenesulfonic acid, salicylic acid and the like. For a description of pharmaceutically acceptable acid addition salts as prodrugs, see Bundgaard, H., supra.

“Pharmaceutically acceptable ester” refers to those esters which retain, upon hydrolysis of the ester bond, the biological effectiveness and properties of the carboxylic acid or alcohol and are not biologically or otherwise undesirable. For a description of pharmaceutically acceptable esters as prodrugs, see Bundgaard, H., ed., Design of Prodrugs, Elsevier Science Publishers, Amsterdam (1985). These esters are typically formed from the corresponding carboxylic acid and an alcohol. Generally, ester formation can be accomplished via conventional synthetic techniques. (See, e.g., March Advanced Organic Chemistry, 3rd Ed., John Wiley & Sons, New York (1985) p. 1157 and references cited therein, and Mark et al. Encyclopedia of Chemical Technology, John Wiley & Sons, New York (1980)). The alcohol component of the ester will generally comprise (i) a C₂-C₁₂ aliphatic alcohol that can or can not contain one or more double bonds and can or can not contain branched carbons or (ii) a C₇-C₁₂ aromatic or heteroaromatic alcohols. This invention also contemplates the use of those compositions which are both esters as described herein and at the same time are the pharmaceutically acceptable acid addition salts thereof.

“Pharmaceutically acceptable amide” refers to those amides which retain, upon hydrolysis of the amide bond, the biological effectiveness and properties of the carboxylic acid or amine and are not biologically or otherwise undesirable. For a description of pharmaceutically acceptable amides as prodrugs, see Bundgaard, H., ed.; Design of Prodrugs, Elsevier Science Publishers, Amsterdam (1985). These amides are typically formed from the corresponding carboxylic acid and an amine. Generally, amide formation can be accomplished via conventional synthetic techniques. (See, e.g., March Advanced Organic Chemistry, 3rd Ed., John Wiley & Sons, New York (1985) p. 1152 and Mark et al. Encyclopedia of Chemical Technology, John Wiley & Sons, New York (1980)) This invention also contemplates the use of those compositions which are both amides as described herein and at the same time are the pharmaceutically acceptable acid addition salts thereof.

“Pharmaceutically or therapeutically acceptable carrier” refers to a carrier medium which does not interfere with the effectiveness of the biological activity of the active ingredients and which is not toxic to the host or patient.

“Stereoisomer” refers to a chemical compound having the same molecular weight, chemical composition, and constitution as another, but with the atoms grouped differently. That is, certain identical chemical moieties are at different orientations in space and, therefore, when pure, has the ability to rotate the plane of polarized light. However, some pure stereoisomers may have an optical rotation that is so slight that it is undetectable with present instrumentation. The compounds of the instant invention may have one or more asymmetrical carbon atoms and therefore include various stereoisomers. All stereoisomers are included within the scope of the invention.

“Therapeutically- or pharmaceutically-effective amount” as applied to the compositions of the instant invention refers to the amount of composition sufficient to induce a desired biological result. That result can be alleviation of the signs, symptoms, or causes of a disease, or any other desired alteration of a biological system. In the present invention, the result will typically involve an increase in red blood cell production.

Amino acid residues in peptides are abbreviated as follows: Phenylalanine is Phe or F; Leucine is Leu or L; Isoleucine is Ile or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Asparcic Acid is Asp or D; Glutamic Acid is Glu or E; Cysteine is Cys or C; Tryptophan is Trp or W; Arginine is Arg or R; and Glycine is Gly or G. Additionally, Bu is Butoxy, Bzl is benzyl, CHA is cyclohexylamine, Ac is acetyl, Me is methyl, Pen is penicillamine, Aib is amino isobutyric acid, Nva is norvaline, Abu is amino butyric acid, Thi is thienylalanine, OBn is O-benzyl, and hyp is hydroxyproline.

In addition to peptides consisting only of naturally-occurring amino acids, peptidomimetics or peptide analogs are also provided. Peptide analogs are commonly used in the pharmaceutical industry as non-peptide drugs with properties analogous to those of the template peptide. These types of non-peptide compound are termed “peptide mimetics” or “peptidomimetics” (Fauchere, J. Adv. Drug Res. 15:29 (1986); Veber and Freidinger TINS p.392 (1985); and Evans et al. J. Med. Chem. 30:1229 (1987), which are incorporated herein by reference). Peptide mimetics that are structurally similar to therapeutically useful peptides may be used to produce an equivalent or enhanced therapeutic or prophylactic effect. Generally, peptidomimetics are structurally similar to a paradigm polypeptide (i.e., a polypeptide that has a biological or pharmacological activity), such as naturally-occurring receptor-binding polypeptide, but have one or more peptide linkages optionally replaced by a linkage selected from the group consisting of: —CH₂NH—, —CH₂S—, —CH₂—CH₂—, —CH═CH—(cis and trans), —COCH₂—, —CH(OH)CH₂—, and —CH₂SO—, by methods known in the art and further described in the following references: Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468 (general review); Hudson, D. et al., Int J Pept Prot Res 14:177-185 (1979) (—CH₂NH—, CH₂CH₂—); Spatola et al. Life Sci 38:1243-1249 (1986) (—CH₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln. EP 45665 CA (1982): 97:39405 (1982) (—CH(OH)CH₂—); Holladay et al. Tetrahedron Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others.

Labeling of peptidomimetics usually involves covalent attachment of one or more labels, directly or through a spacer (e.g., an amide group); to non-interfering position(s) on the peptidomimetic that are predicted by quantitative structure-activity data and/or molecular modeling. Such non-interfering positions generally are positions that do not form direct contacts with the macromolecules(s) (e.g., immunoglobulin superfamily molecules) to which the peptidomimetic binds to produce the therapeutic effect. Derivitization (e.g., labeling) of peptidomimetics should not substantially interfere with the desired biological or pharmacological activity of the peptidomimetic. Generally, peptidomimetics of receptor-binding peptides bind to the receptor with high affinity and possess detectable biological activity (i.e., are agonistic or antagonistic to one or more receptor-mediated phenotypic changes).

Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference); for example, by adding internal cysteine residues capable of forming intramolecular disulfide bridges which cyclize the peptide.

Synthetic or non-naturally occurring amino acids refer to amino acids which do not naturally occur in vivo but which, nevertheless, can be incorporated into the peptide structures described herein. Preferred synthetic amino acids are the D-a-amino acids of naturally occurring L-a-amino acid as well as non-naturally occurring D- and L-a-amino acids represented by the formula H₂NCHR⁵COOH where R⁵ is 1) a lower alkyl group, 2) a cycloalkyl group of from 3 to 7 carbon atoms, 3) a heterocycle of from 3 to 7 carbon atoms and 1 to 2 heteroatoms selected from the group consisting of oxygen, sulfur, and nitrogen, 4) an aromatic residue of from 6 to 10 carbon atoms optionally having from 1 to 3 substituents on the aromatic nucleus selected from the group consisting of hydroxyl, lower alkoxy, amino, and carboxyl, 5) -alkylene-Y where alkylene is an alkylene group of from 1 to 7 carbon atoms and Y is selected from the group consisting of (a) hydroxy, (b) amino, (c) cycloalkyl and cycloalkenyl of from 3 to 7 carbon atoms, (d) aryl of from 6 to 10 carbon atoms optionally having from 1 to 3 substituents on the aromatic nucleus selected from the group consisting of hydroxyl, lower alkoxy, amino and carboxyl, (e) heterocyclic of from 3 to 7 carbon atoms and 1 to 2 heteroatoms selected from the group consisting of oxygen, sulfur, and nitrogen, (f) —C(O)R² where R² is selected from the group consisting of hydrogen, hydroxy, lower alkyl, lower alkoxy, and —NR³R⁴ where R³ and R⁴are independently selected from the group consisting of hydrogen and lower alkyl, (g) —S(O)ₙR₆ where n is an integer from 1 to 2 and R₆ is lower alkyl and with the proviso that R₅ does not define a side chain of a naturally occurring amino acid.

Other preferred synthetic amino acids include amino acids wherein the amino group is separated from the carboxyl group by more than one carbon atom such as b-alanine, g-aminobutyric acid, and the like.

Particularly preferred synthetic amino acids include, by way of example, the. D-amino acids of naturally occurring L-amino acids, L-1-napthyl-alanine, L-2-naphthylalanine, L-cyclohexylalanine, L-2-amino isobutyric acid, the sulfoxide and sulfone derivatives of methionine (i.e., HOOC—(H₂ NCH)CH₂ CH₂—S(O)ₙR₆) where n and R₆ are as defined above as well as the lower alkoxy derivative of methionine (i.e., HOOC—(H₂ NCH)CH₂ CH₂—OR⁶ where R⁶ is as defined above).

“Detectable label” refers to materials, which when covalently attached to the peptides and peptide mimetics of this invention, permit detection of the peptide and peptide mimetics in vivo in the patient to whom the peptide or peptide mimetic has been administered. Suitable detectable labels are well known in the art and include, by way of example, radioisotopes, fluorescent labels (e.g., fluorescein), and the like. The Darticular detectable label employed is not critical and is selected relative to the amount of label to be employed as well as the toxicity of the label at the amount of label employed. Selection of the label relative to such factors is well within the skill of the art.

Covalent attachment of the detectable label to the peptide or peptide mimetic is accomplished by conventional methods well known in the art. For example, when the ¹²⁵I radioisotope is employed as the detectable label, covalent attachment of ¹²⁵I to the peptide or the peptide mimetic can be achieved by incorporating the amino acid tyrosine into the peptide or peptide mimetic and then iodating the peptide. If tyrosine is not present in the peptide or peptide mimetic, incorporation of tyrosine to the N or C terminus of the peptide or peptide mimetic can be achieved by well known chemistry. Likewise, ³²P can be incorporated onto the peptide or peptide mimetic as a phosphate moiety through, for example, a hydroxyl group on the peptide or peptide mimetic using conventional chemistry.

The present invention provides compounds that bind to and activate the TPO-R or otherwise behave as a TPO agonist. These compounds include “lead” peptide compounds and “derivative” compounds constructed so as to have the same or similar molecular structure or shape as the lead compounds but that differ from the lead compounds either with respect to susceptibility to hydrolysis or proteolysis and/or with respect to other biological properties, such as increased affinity for the receptor. The present invention also provides compositions comprising an effective amount of a TPO agonist, and more particularly a compound, that is useful for treating anemia.

Peptides having a binding affinity to TPO-R can be readily identified by random peptide diversity generating systems coupled with an affinity enrichment process. This process is disclosed in U.S. Pat. Nos. 6,251,864, 6,083,913, 6,121,238, 5,932,546, 5,869,451, 6,506,362, and 6,465,430, and in Cwirla et al., Proc. Natl. Acad. Sci. USA 87:6378-6382 (1990).

The compounds of the invention can also be administered to warm blooded-animals, including humans, to activate the TPO-R in vivo. Thus, the present invention encompasses methods for therapeutic treatment of anemia that comprise administering a compound of the invention in amounts sufficient to mimic the effect of TPO on TPO-R in vivo.

The activity of the compounds of the present invention can be evaluated either in vitro or in vivo in one of the numerous models described in McDonald Am. J. of Pediatric Hematology/Oncology 14:8-21 (1992), which is incorporated herein by reference.

According to one embodiment, the compositions of the present invention are useful for treating anemia associated with bone marrow transfusions, radiation therapy, or chemotherapy. The compounds typically will be administered prophylactically prior to chemotherapy, radiation therapy, or bone marrow transplant or after such exposure.

Accordingly, the present invention also provides pharmaceutical compositions comprising, as an active ingredient, at least one of the peptides or peptide mimetics of the invention in association with a pharmaceutical carrier or diluent. The compounds of this invention can be administered by oral, pulmonary, parental (intramuscular, intraperitoneal, intravenous (IV) or subcutaneous injection), inhalation (via a fine powder formulation), transdermal, nasal, vaginal, rectal, or sublingual routes of administration and can be formulated in dosage forms appropriate for each route of administration. See, e.g., Bernstein et al. PCT Patent Publication No. WO 93/25221; Pitt et al. PCT Patent Publication No. WO 94/17784; and Pitt et al. European Patent Application 613,683, each of which is incorporated herein by reference.

Solid dosage forms for oral administration include capsules, tablets, pills, powders, and granules. In such solid dosage forms, the active compound is admixed with at least one inert pharmaceutically acceptable carrier such as sucrose, lactose, or starch. Such dosage forms can also comprise, as is normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration include pharmaceutically acceptable emulsions, solutions, suspensions, syrups, with the elixirs containing inert diluents commonly used in the art, such as water. Besides such inert diluents, compositions can also include adjuvants, such as wetting agents, emulsifying and suspending agents, and sweetening, flavoring, and perfuming agents.

Preparations according to this invention for parental administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

Compositions for rectal or vaginal administration are preferably suppositories which may contain, in addition to the active substance, excipients such as cocoa butter or a suppository wax. Compositions for nasal or sublingual administration are also prepared with standard excipients well known in the art.

The compositions containing the compounds can be administered for prophylactic and/or therapeutic treatments. In therapeutic applications, compositions are administered to a patient already suffering from a disease, as described above, in an amount sufficient to cure or at least partially arrest the symptoms of the disease and its complications. An amount adequate to accomplish this is defined as “therapeutically effective dose”. Amounts effective for this use will depend on the severity of the disease and the weight and general state of the patient.

The compositions of the invention can also be microencapsulated by, for example, the method of Tice and Bibi (in Treatise on Controlled Drug Delivery, ed. A. Kydonieus, Marcel Dekker, N.Y. (1992), pp. 315-339).

In prophylactic applications, compositions containing the compounds of the invention are administered to a patient susceptible to-or otherwise at risk of a particular disease. Such an amount is defined to be a “prophylactically effective dose”. In this use, the precise amounts again depend on the patient's state of health and weight.

The quantities of the TPO agonist necessary for effective therapy will depend upon many different factors, including means of administration, target site, physiological state of the patient, and other medicants administered. Thus, treatment dosages should be titrated to optimize safety and efficacy. Typically, dosages used in vitro may provide useful guidance in the amounts useful for in situ administration of these reagents. Animal testing of effective doses for treatment of particular disorders will provide further predictive indication of human dosage. Various considerations are described, e.g., in Gilman et al. (eds), Goodman and Gilman's: The Pharmacological Basis of Therapeutics, 8th ed., Pergamon Press (1990); and Remington's Pharmaceutical Sciences, 7th ed., Mack Publishing Co., Easton, Pa. (1985); each of which is hereby incorporated by reference.

The peptides and peptide mimetics of this invention are effective in treating anemia when administered at a dosage range of from about 1 ug to about 300 ug/kg of body weight per day. The specific dose employed is regulated by the route of administration as well as by the judgment of the attending clinician depending upon factors such as the severity of the condition, the age and general condition of the patient, and the like.

### EXAMPLES

The effects of Compound I on mice treated with carboplatin were observed. For all examples herein, a 10 mg/ml stock solution of Compound I was prepared in sterile saline. For mixing, the preparation was placed on a gyratory shaker for 15 min at 200 rpm. This method was used to dissolve Compound I without foaming. The stock was filtered using a GV Millex (0.22um) filter. Dosing solutions were then prepared from this stock using sterile saline. The stock and dosing solutions were prepared fresh on the day of use.

### Example 1

The effect of Compound I on the duration and severity of anemia following treatment of mice with carboplatin as determined by changes in hemoglobin levels, red blood cell count and hematocrit, was observed. For this study, increasing amounts of Compound I were administered to mice one day following a carboplatin dose to characterize a possible dose-dependent effect on various red blood cell parameters.

The groups of mice were treated with either carboplatin or vehicle (Phosphate Buffered Saline, PBS ) by intraperitoneal administration on Days −2 and −1 as delineated below. The optimal dose of carboplatin used to induce thrombocytopenia in the BALB/c mouse strain was previously determined to be a fractionated total dose of 120mg/kg given as two consecutive daily injections (i.e., 2×60 mg/kg). One day following the second dose of carboplatin, groups of mice were treated with Compound I or vehicle (sterile saline, SS, preservative-free 0.9% sodium chloride ) by iv (bolus) injection as delineated in Table 1. The dose was administered on a per-weight basis (100 ul/10 g body weight).

TABLE 1
Treatment Groups:

| | | Pretreatment | | | |
|---|---|---|---|---|---|
| | | (ip), | | Dose (iv) | |
| Gp | N | Day −2 & −1 | Test Article | Day 0 | Blood Collection |
| 1 | 10 | Vehicle (PBS) | Vehicle (SS) | Sham | Sac 5 mice on Day 5 & 11 |
| 2 | 20 | Carboplatin | Vehicle (SS) | Sham | Sac 5 mice on Days 5, 7, 9 & 11 |
| 3 | 20 | Carboplatin | Compound I | 300 ug/kg | Sac 5 mice on Days 5, 7, 9 & 11 |
| 4 | 20 | Carboplatin | Compound I | 1000 ug/kg | Sac 5 mice on Days 5, 7, 9 & 11 |
| 5 | 20 | Carboplatin | Compound I | 3000 ug/kg | Sac 5 mice on Days 5, 7, 9 & 11 |

Gp = Group;
Sac = Sacrifice

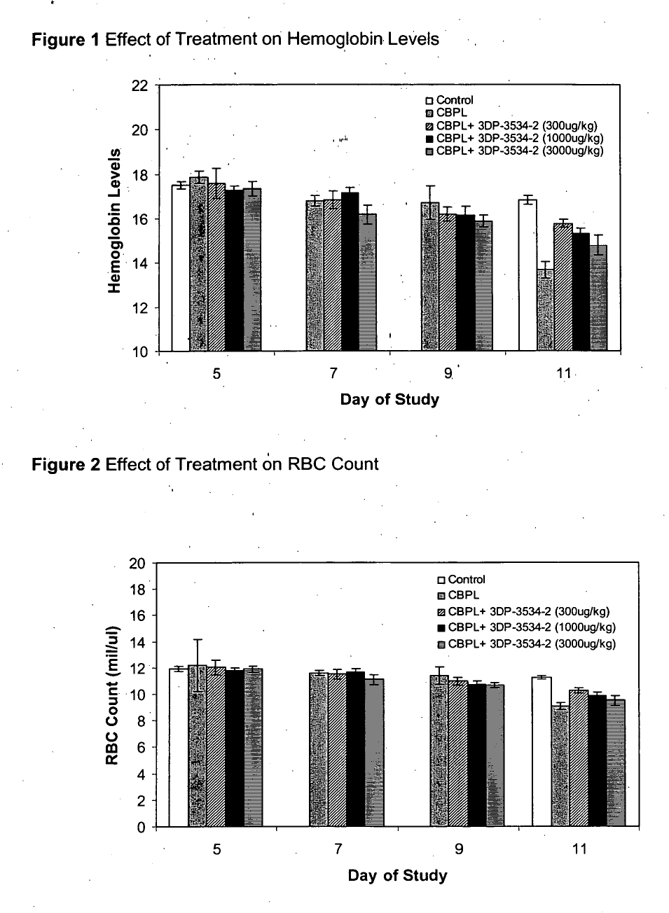

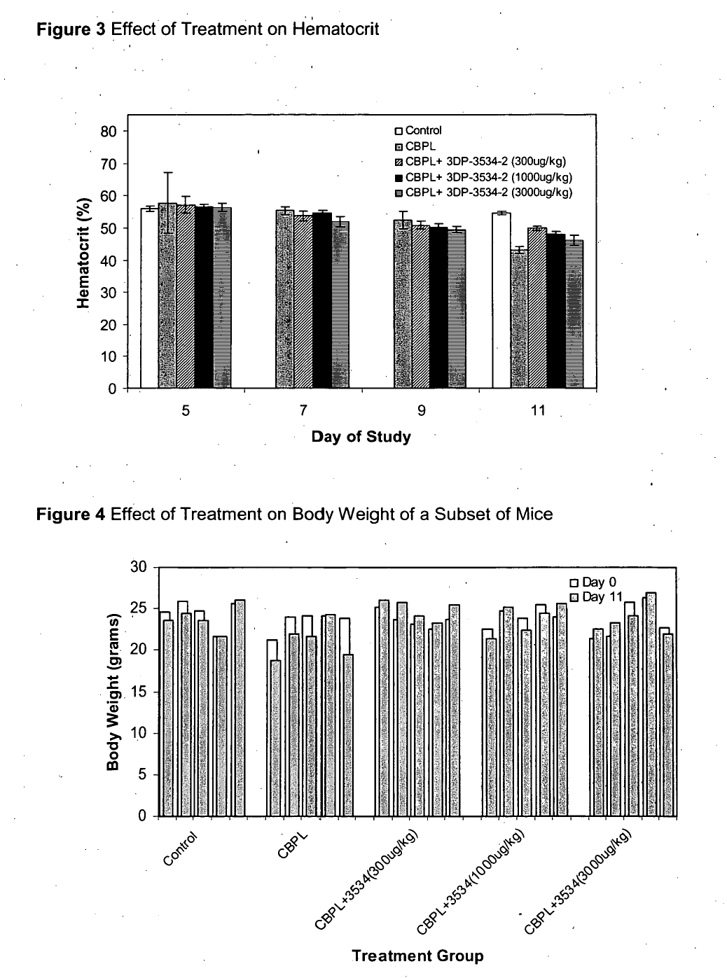

On Days 5, 7, 9 and 11, five mice in each test group were weighed and then sacrificed using CO₂-asphyxiation and exsanguinations via cardiac puncture. The blood samples were transferred to separate EDTA (lavender-top) microcontainers for hematologic evaluation. Groups of control mice (5) were processed on Days 5 and 11. Results are shown in FIGS. 1-4. The data are presented graphically as group means+SEM.

Treatment of mice with carboplatin alone caused about a 20% decrease in hemoglobin levels in the mice by Day 11. This decrease was inhibited by treatment with all doses of Compound I. Minor decreases in RBC count and hematocrit were also associated with carboplatin treatment, an effect that was inhibited by treatment with Compound I; however statistical evaluation of this effect was not conducted. Mice in all groups treated with carboplatin alone or carboplatin plus the various doses of Compound I experienced weight loss on Days 5, 7 and 9 relative to body weight measurements collected on Day 0. Analysis of the body weight measurements in a subset of mice over the 11-Day study period suggests that carboplatin treatment alone caused the observed decrease in body weights and that Compound I enhanced the recovery of the lost body weight at all doses tested.

Mice treated with carboplatin alone began to exhibit altered appearance and behavior by Day 5. Some of the mice assumed a hunched position and appeared flaccid. Many mice also had soiled anogenital areas. Treatment with Compound I decreased the onset, frequency and severity of these signs in manner that appeared to be dose-dependent.

### Example 2

The possibility that Compound I may sensitize bone marrow hematopoietic stem cells of mice to the toxic effects of carboplatin treatment was examined. For this study, a dose of Compound I was administered to the mice seven days prior to the carboplatin dose or immediately after carboplatin treatment. An additional group was treated with Compound I both prior to and after carboplatin administration. The effect of these dosing regimens on hematological parameters was also observed.

The groups of mice were treated with either carboplatin or vehicle (Phosphate Buffered Saline, PBS) by ip administration on Days 7 and 8 as delineated below. The optimal dose of carboplatin used to induce thrombocytopenia in the BALB/c mouse strain was previously determined to be a fractionated total dose of 120 mg/kg given as two consecutive daily injections (i.e., 2×60 mg/kg). Seven days prior to the first carboplatin dose or one (1) hour after the second dose of carboplatin, groups of mice were treated with Compound I (300 ug/kg) or vehicle (sterile saline, SS, preservative-free 0.9% sodium chloride) by iv (bolus) injection as delineated in Table 2. An additional group was treated with Compound I both before (Day 0) and after (Day 8, t=1 h) the carboplatin dose. All dosing was performed on a per-weight basis (100 ul/10 g body weight).

TABLE 2
Study Design:

| | | | Carboplatin | | |
|---|---|---|---|---|---|
| | | | (CBPL) | Dose (iv) | |
| | | | [60 mg/kg, | Day 8 | Blood Collection |
| | | Dose (iv) | q2d], (ip), | (1 h after 2^{nd} | Sac 5 mice on |
| Gp | N | Day 0 | Day 7 & 8 | CBPL dose) | Days |
| 1 | 10 | Vehicle (*SS) | Vehicle | Vehicle | 14 & 26 |
| | | | (PBS) | (*SS) | |
| 2 | 20 | Vehicle (*SS) | Carboplatin | Vehicle | 14, 18, 22 & 26 |
| | | | | (*SS) | |
| 3 | 20 | Compound I | Carboplatin | Vehicle | 14, 18, 22 & 26 |
| | | 300 ug/kg | | (*SS) | |
| 4 | 20 | Vehicle (*SS) | Carboplatin | Compound I | 14, 18, 22 & 26 |
| | | | | 300 ug/kg | |
| 5 | 20 | Compound I | Carboplatin | Compound I | 14, 18, 22 & 26 |
| | | 300 ug/kg | | 300 ug/kg | |

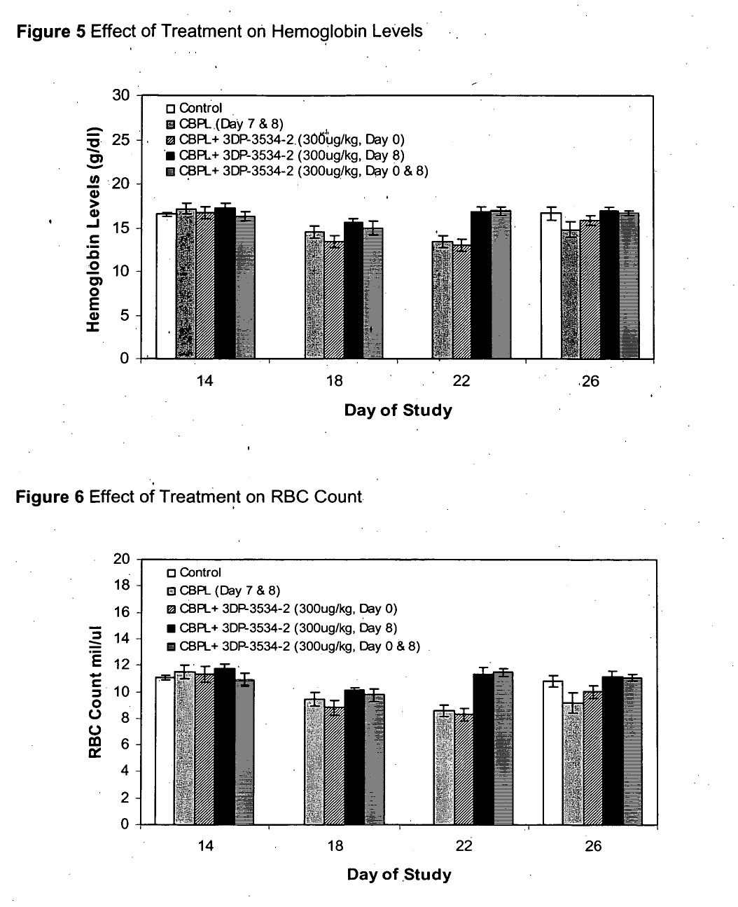

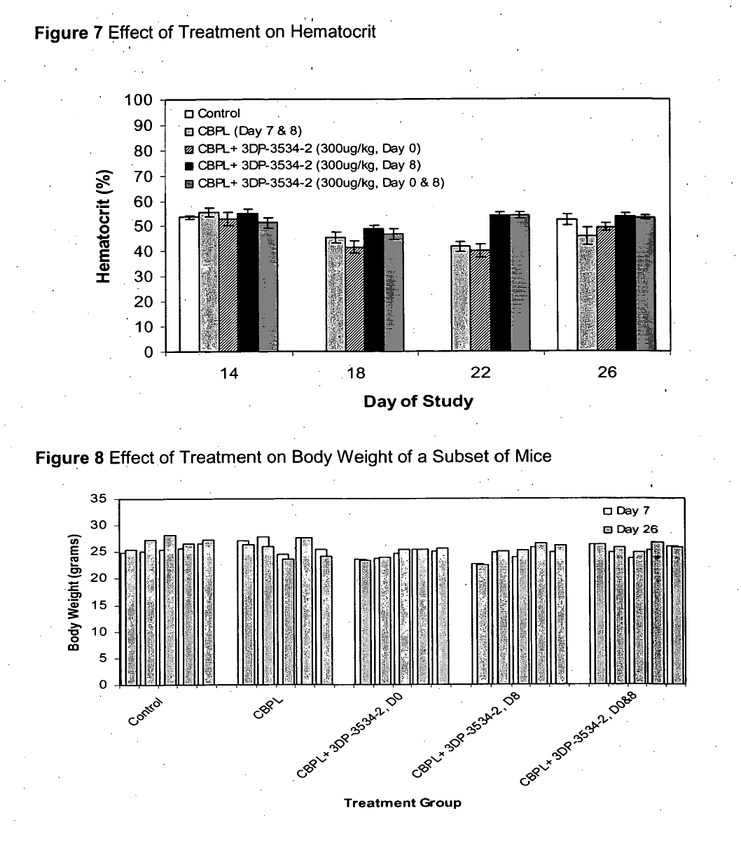

On Days 14, 18, 22 and 26, five mice in each test group were weighed and then sacrificed using CO₂-asphyxiation and exsanguination via cardiac puncture. The blood samples were transferred to separate EDTA (lavender-top) microcontainers for hematological evaluation. Groups of control mice (5) were processed on Days 14 and 26. Results are shown in FIGS. 5-8. The data are presented graphically as group means+SEM.

Treatment of mice with carboplatin alone caused decreases (approx. 18%) in hemoglobin levels, RBC counts and hematocrits in the surviving mice by Days 18 and 22 compared to control groups. These decreases were prevented by the administration of Compound I on Day 8 (1 hour after the second carboplatin treatment) without or with an additional dose of Compound I on Day 0; however, the administration of Compound I on Day 0 (only) failed to affect carboplatin-induced changes in these erythrocyte parameters.

All mice in the control group experienced normal weight gain between Days 7 and 26, while all mice treated with carboplatin alone lost small amounts of body weight (averaging approximately 4%) during the same time period. Mice in all groups that were treated with carboplatin and various co-treatments with Compound I either maintained body weight or experienced normal weight gain between Days 7 and 26. Analysis of the body weight measurements over the study period suggests that carboplatin treatment was the major contributor to the observed decreases in body weights and that co-treatment with Compound I prevented this weight loss, however a statistical analysis was not conducted. Differences in body weights observed between Day 7 (prior to dosing with carboplatin) and Day 26 (study termination) are presented in FIG. 10.

All mice in the control groups appeared normal throughout the study period. Mice treated with carboplatin alone began to exhibit altered appearance and behavior as early as Day 12 with frequent signs of hunching and appearing unkempt. Many mice receiving carboplatin (without or with Compound I treatment) assumed a hunched position and appeared unkempt during the latter half of the study period. Treatment with Compound I on Day 8 without or with additional treatment on Day 0 appeared to delay the onset of these signs and treatment on Days 0 and 8 decreased the severity and duration as well; however a detailed analysis of the effects of treatment on systemic observations was not conducted.

### Example 3

The effect of Compound I on the duration and severity of anemia following dosing regimens in which Compound I is administered at various times following the carboplatin treatment was observed. For this study, an amount of Compound I was administered to mice, one (1) hour, one (1) day or four (4) days following a carboplatin dose.

The groups of mice were treated with either carboplatin) or vehicle (Phosphate Buffered Saline, PBS) by ip administration on Days −1 and 0 as delineated below. The optimal dose of carboplatin used to induce thrombocytopenia in the BALB/c mouse strain was previously determined to be a fractionated total dose of 120 mg/kg given as two consecutive daily injections (i.e., 2×6 Omg/kg). One hour (Day 0), one day (Day 1) or four days (Day 4) following the second dose of carboplatin, groups of mice were treated with Compound I (300 ug/kg) or vehicle (sterile saline, SS, preservative-free 0.9% sodium chloride) by iv (bolus) injection as delineated in Table 3. The dose was administered on a per-weight basis (100 ul/10g body weight).

TABLE 3
Treatment Groups:

| | | Pretreatment | | | |
|---|---|---|---|---|---|
| | | [2 × 60 | | | |
| | | mg/kg], (ip), | | | Blood Collection |
| Gp | N | Day −1 & 0 | Test Article | Dose (iv) | Sac 5 mice on |
| 1 | 10 | Vehicle (PBS) | Vehicle (SS) | Sham | Days 6 & 12 |
| 2 | 20 | Carboplatin | Vehicle (SS) | Sham | Days 6, 8, 10 & 12 |
| 3 | 20 | Carboplatin | Compound I | 300 ug/kg, | Days 6, 8, 10 & 12 |
| | | | | Day 0 | |
| 4 | 20 | Carboplatin | Compound I | 300 ug/kg, | Days 6, 8, 10 & 12 |
| | | | | Day 1 | |
| 5 | 20 | Carboplatin | Compound I | 300 ug/kg, | Days 6, 8, 10 & 12 |
| | | | | Day 4 | |

Gp = Group;
Sac = Sacrifice

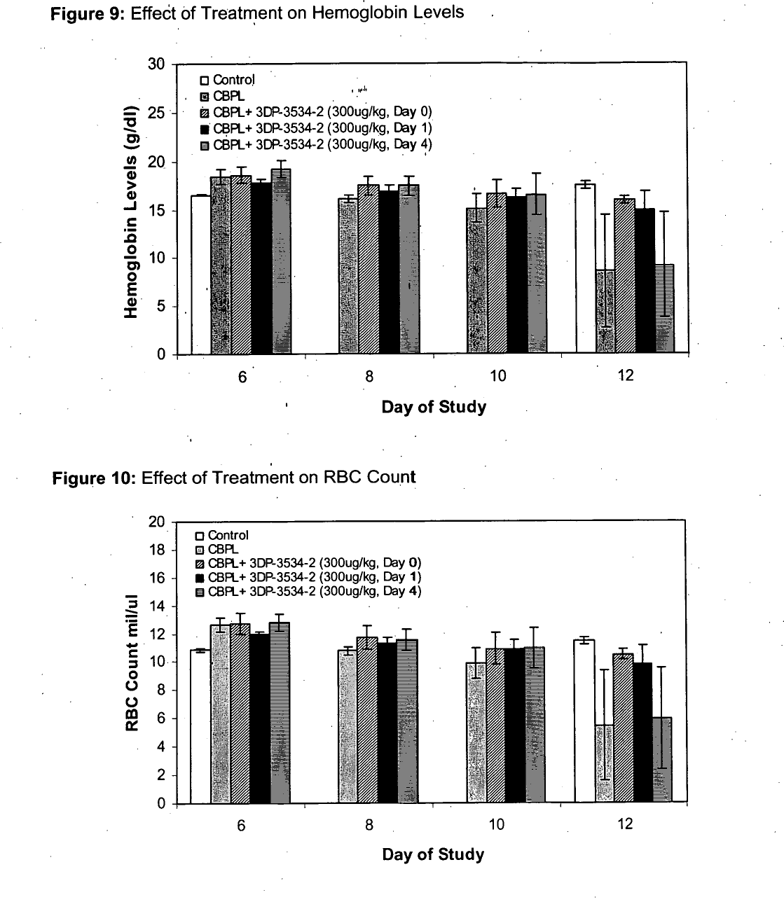

On Days 6, 8, 10 & 12, five mice in each test group were weighed and then sacrificed using CO₂-asphyxiation and exsanguinations via cardiac puncture. The blood samples were transferred to separate EDTA (lavender-top) microcontainers for hematological evaluation. Groups of control mice (5) were processed on Days 6 & 12. Results are shown in FIGS. 9-12. The data are presented graphically as group means+SEM.

Treatment of mice with carboplatin alone caused dramatic decreases (approx. 47%) in hemoglobin levels, RBC counts and hematocrits in the surviving mice (2 mice) by Day 12 compared to control groups. These decreases were prevented by the administration of Compound I on Day 0 (1 hour after carboplatin treatment) and on Day 1; however, the administration of Compound I on Day 4 failed to affect carboplatin-induced changes in these erythrocyte parameters.

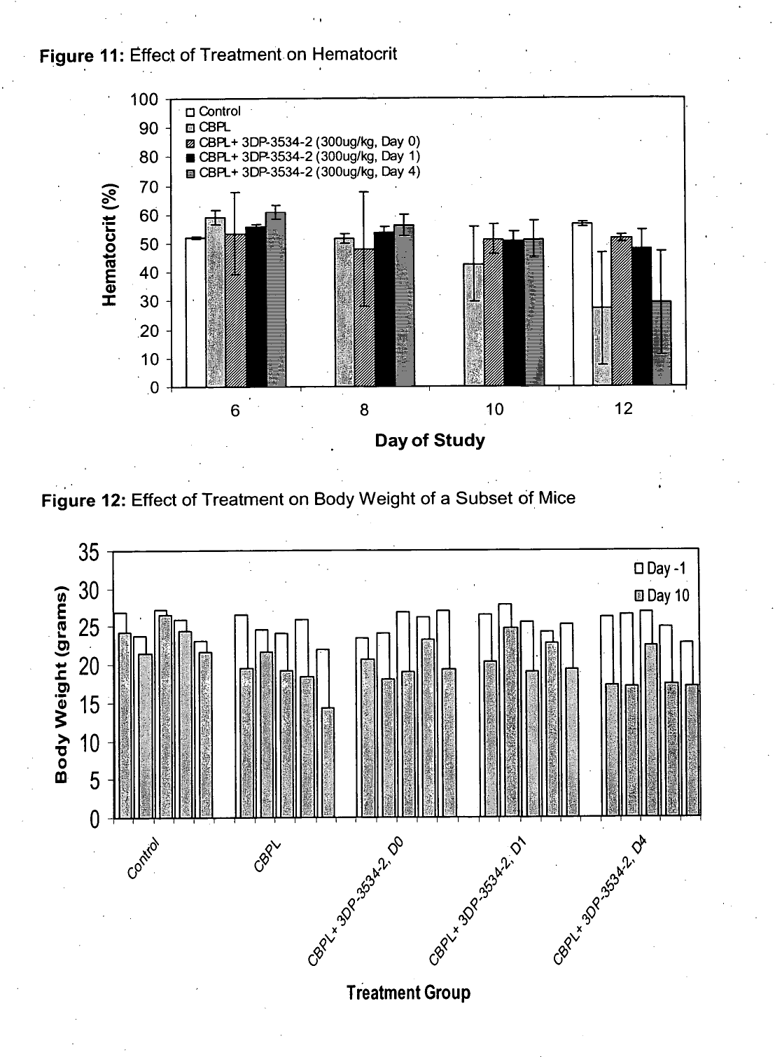

Mice in all groups treated with carboplatin alone or carboplatin plus the various doses of Compound I experienced weight loss on Days 6, 8, 10 and 12 relative to body weight measurements collected on Day −-1. Analysis of the body weight measurements over the study period suggests that carboplatin was the major contributor to the observed decreases in body weights. The administration of Compound I on Days 0, Day 1 or Day 4 did not appear to affect the weight loss associated with carboplatin treatment, however a statistical analysis was not conducted. Decreases in body weights observed between Day −1 and Day 10 are presented in FIG. 11).

All mice in the control groups appeared normal throughout the study period. Mice treated with carboplatin alone began to exhibit altered appearance and behavior as early as Day 2 with frequent signs of hunching and appearing flaccid. Many mice receiving carboplatin (without or with Compound I treatment) assumed a hunched position and appeared flaccid in the. latter half of the study period. Some of these mice also had soiled anogenital areas. Other infrequent signs included appearing emaciated, having sagging eyelids and exhibiting an abnormal gate. Treatment with Compound I did not appear to have a dramatic effect on the onset, frequency or the severity of these signs, however a detailed analysis was not conducted.

Prevention of carboplatin-induced anemia is observed when animals are dosed with Compound I within 24 hours of chemotherapy. This data suggests that Compound I has myeloprotective effects that are not limited to the megakaryocyte lineage.

### Example 4

The ability of Compound I to function as a survival factor for megakaryocyte and erythrocyte lineages in carboplatin-treated mice as determined by changes in hematological parameters was observed. In previous studies, doses of Compound I as low as 300 ug/kg were found to prevent the anemia induced by carboplatin. In this study, the effect of lower doses Compound I (i.e., 30, 100 and 300 ug/kg) on the survival of erythrocyte lineages was examined to characterize the dose-response for this effect.

The groups of mice were treated with either carboplatin or vehicle (Phosphate Buffered Saline, PBS) by ip administration on Days −1 and 0 as delineated below. The optimal dose of carboplatin used to induce thrombocytopenia in the BALB/c mouse strain was previously determined to be a fractionated total dose of 120mg/kg given as two consecutive daily injections (i.e., 2×60 mg/kg). Approximately one hour following the second dose of carboplatin, groups of mice were treated with Compound I or vehicle (sterile saline, SS, preservative-free 0.9% sodium chloride) by iv (bolus) injection as delineated in Table 4. The dose was administered on a per-weight basis (100ul/10 g body weight).

TABLE 4
Treatment Groups:

| | | Pretreatment | | | |
|---|---|---|---|---|---|
| | | (ip), | | Dose (iv) | |
| Gp | N | Day −1 & 0 | Test Article | Day 0 | Blood Collection |
| 1 | 10 | Vehicle (PBS) | Vehicle (SS) | Sham | Sac 5 mice on Day |
| | | | | | 6 & 12 |
| 2 | 15 | Carboplatin | Vehicle (SS) | Sham | Sac 5 mice on Days |
| | | | | | 6, 8 & 12 |
| 3 | 15 | Carboplatin | Compound I | 30 ug/kg | Sac 5 mice on Days |
| | | | | | 6, 8 & 12 |
| 4 | 15 | Carboplatin | Compound I | 100 ug/kg | Sac 5 mice on Days |
| | | | | | 6, 8 & 12 |
| 5 | 15 | Carboplatin | Compound I | 300 ug/kg | Sac 5 mice on Days |
| | | | | | 6, 8 & 12 |

Gp = Group;
Sac = Sacrifice

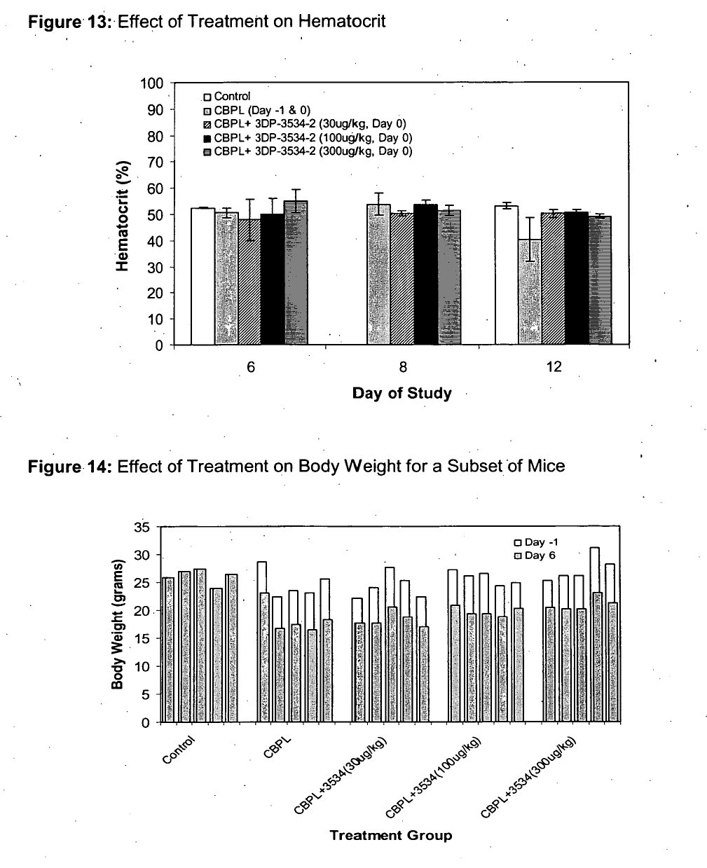

On Days 6, 8 and 12, five mice in each test group were weighed and then sacrificed using CO²-asphyxiation and exsanguinations via cardiac puncture. The blood samples were transferred to separate EDTA (lavender-top) microcontainers for hematologic evaluation. Groups of control mice (5) were processed on Days 6 & 12. Results are shown in FIGS. 13-14.

Treatment of mice with carboplatin alone caused a greater than 25% decrease in hemoglobin levels in the mice by Day 12. This decrease was totally inhibited by treatment with all doses of Compound I. Compound I also effectively inhibited the decreases in RBC counts and hematocrit that were induced by carboplatin treatment.

Essentially all of the mice in all groups treated with either carboplatin alone or carboplatin plus the various doses of Compound I experienced weight loss on Days 6, 8 and 12 relative to body weight measurements collected on Day −1. Analysis of the body weight measurements over the 13-Day study period indicates that carboplatin treatment alone caused the observed decrease in body weights. Compound I did not appear to affect weight loss or recovery in this study.

Mice treated with carboplatin alone began to exhibit altered appearance and behavior by Day 4. Some of the mice assumed a hunched position and appeared unkempt. Many mice also had soft stool. Few animals appeared flaccid and few presented with blood in stool. Treatment with Compound I decreased the onset, frequency and severity of these signs in manner that appeared to be dose-dependent.

Compound I functioned to maintain the survival of erythrocyte lineages in carboplatin-treated mice as determined by peripheral blood platelet counts and other hematological parameters. All doses of Compound I were found to completely prevent the anemia induced by carboplatin on Day 12. These results suggest a differential sensitivity/responsiveness of the megakaryocyte and erythrocyte lineages to the “survival maintenance” effects of Compound I.

### Example 5

Groups of mice were treated with two rounds of the chemotherapeutic agent (carboplatin) ten days apart, with each round consisting of two consecutive days of carboplatin (i.e., 70 mg/kg/day administered on Days −1 & 0 and Days 10 & 11) as delineated below. The dose of carboplatin utilized for these survival studies exceeded the maximal tolerated dose for mice (i.e., 120 mg/kg; administered as 60 mg/kg/day on 2 consecutive days). One hour following the second dose of carboplatin in each round (i.e., Day 0 and 11) mice were treated with Compound I (100 ug/kg) or vehicle (sterile saline, SS, preservative-free 0.9% sodium chloride) by iv (bolus) injection as delineated below. The dose was administered on a per weight basis (100 ul/10 g body weight).

Study Design:

| | | Pretreatment | | | |
|---|---|---|---|---|---|
| | | (ip), Day −1 | Test Article | Dose (iv) | Blood Collection/ |
| | | & 0, | 100 ug/kg | ˜1 h after 2nd CBPL | Analysis |
| Gp | N | Day 10 & 11 | (iv) | dose for ea. cycle | Sac 5 mice on |
| 1 | 25 | Vehicle (PBS) | Vehicle | Sham, Day 0 & 11 | Days 7, 10, 18, 21 & |
| | | | (*SS) | | 28 |
| 2 | 25 | Carboplatin | Vehicle | Sham, Day 0 & 11 | Days 7, 10, 18, 21 & |
| | | (70 mg/kg) | (*SS) | | 28 |
| 3 | 25 | Carboplatin | JNJ26366821 | 100 ug/kg, Day 0 & 11 | Days 7, 10, 18, 21 & |
| | | (70 mg/kg) | | | 28 |

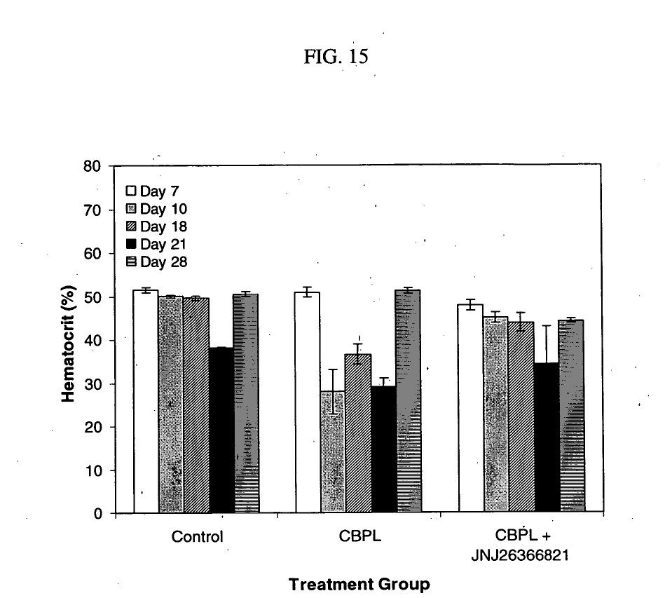

On days 7, 10, 18, 21 and 28 five mice in each test group (25 mice/group) then sacrificed using CO₂-asphyxiation and exsanguinations via cardiac puncture. The blood samples were transferred to separate EDTA (lavender-top) microcontainers for hematological evaluation. Groups of control mice treated with the vehicles alone were processed in the same manner. Results are shown in FIG. 15. Treatment of mice with two rounds of carboplatin resulted in the development of a moderate anemia that was observed between days 10 and 21 while, mice treated with 2 rounds of carboplatin and Compound I maintained hematocrit values throughout this period that were similar to the control group. Interestingly, of the mice not utilized for hematological evaluation, 7 mice in the group treated with carboplatin alone died between days 4 and 18 while only 1 mouse in the group receiving combination therapy expired within the same period, with most of the deaths occurring within the period of anemia. These results suggest that carboplatin-induced anemia may contribute the death of mice receiving high levels of chemotherapy and that Compound I may function to increase the survival of the mice by preventing the development of the anemia.

Although only preferred embodiments of the invention are specifically described above, it will be appreciated that modifications and variations of the invention are possible without departing from the spirit and intended scope of the invention.

What is claimed is:

1. A method of preventing the development of anemia following treatment comprising administering an effective amount of a TPO mimetic compound to a subject in need thereof.

2. The method of claim 1, wherein said treatment is selected from the group consisting of treatment with cytotoxic agents, anti-tumor agents and radiation.

3. A method of optimizing the production of red blood cells comprising administering an effective amount of a TPO mimetic compound to a subject in need thereof.

4. The method of claim 1, wherein the subject is a human.

5. The method of claim 1, wherein the TPO mimetic compound has reduced immunogenicity relative to one or more of rhTPO and rhIL-11.

6. The method of claim 1 wherein the TPO mimetic compound has an improved PK profile relative to one or more of rhTPO and rhIL-11.

7. The method of claim 1, wherein said effective amount is from about 1 ug to about 300 ug/kg of body weight per day.

8. A pharmaceutical composition for increasing red blood cell production comprising a TPO mimetic compound in admixture with a pharmaceutically acceptable carrier.

9. A method of treating anemia, comprising a step of administering an effective amount of a TPO mimetic compound to a subject in need thereof.

10. A pharmaceutical composition for treating anemia, comprising a TPO mimetic compound in admixture with a pharmaceutically acceptable carrier.

11. The method of claim 1, wherein said red blood cell is selected from the group consisting of lineage specific erythrocyte precursor cell, reticulocyte and erythrocyte.
